(19) United States
(12) Patent Application Publication
(10) Pub. No.: US 2026/0137721 A1
(43) Pub. Date: May 21, 2026

(54) EXTRACELLULAR VESICLES DERIVED FROM HEMATOPOIETIC STEM CELLS OR HEMATOPOIETIC PROGENITOR CELLS AND USES THEREOF

(71) Applicant: Albert Einstein College of Medicine, Bronx, NY (US)

(72) Inventors: Keisuke Ito, Bronx, NY (US); Claudia Morganti, Bronx, NY (US); Massimo Bonora, Ferrara (IT); Barbara Zavan, Ferrara (IT); Paolo Pinton, Ferrara (IT)

(21) Appl. No.: 19/386,450

(22) Filed: Nov. 12, 2025

Related U.S. Application Data
(60) Provisional application No. 63/720,812, filed on Nov. 15, 2024.

(51) Int. Cl. A61K 35/28; C12N 5/0789

(52) U.S. Cl. CPC A61K 35/28; C12N 5/0647; C12N 2501/125; C12N 2501/145; C12N 2501/70; C12N 2501/999; C12N 2502/03

(57) ABSTRACT

Provided herein are methods for expanding populations of hematopoietic stem cells (HSCs) or hematopoietic progenitor cells (HPCs) using extracellular vesicles isolated from hematopoietic stem cells (HSC-derived EVs) or from HPCs (HPC-derived EVs). Also provided are methods of using the isolated HSC-derived EVs and/or HPC-derived EVs as well as the HSCs and HPCs generated by or exposed to the methods disclosed herein.

### CROSS REFERENCE TO RELATED APPLICATIONS

This application claims priority to U.S. Provisional Application No. 63/720,812, filed on Nov. 15, 2024, which is incorporated herein by reference in its entirety.

### STATEMENT REGARDING FEDERALLY SPONSORED RESEARCH

This invention was made with government support under R01DK98263, R01DK115577, and R01HL148852 awarded by the National Institutes of Health (NIH). The government has certain rights in the invention.

### FIELD OF THE INVENTION

The present disclosure relates generally to the field of molecular biology and medicine. More particularly, the methods and compositions herein are useful for treating disease.

### BACKGROUND

The maintenance of the hematopoietic system relies on primitive pluripotent hematopoietic stem cells (HSCs) that have the capacity to self-renew and repopulate all the blood cell lineages with relevant progenitor cells. Due to their capacity for self-renewal and their multipotent, long term reconstituting potential, HSCs have long been considered ideal for transplantation to reconstitute the hematopoietic system after treatment for various hematologic disorders or as a target for the delivery of therapeutic genes. Additionally, human HSCs have potential applications for restoring the immune system in autoimmune diseases and in the induction of tolerance for allogenic solid organ transplantation.

Stem cell transplantation (SCT) is a curative treatment for many blood-related diseases, particularly blood cancers and hereditary blood disorders (e.g., primary immunodeficiencies). SCT involves depleting the patient's diseased bone marrow and hematopoietic system through preconditioning (currently achieved using genotoxic chemotherapy and/or radiotherapy) and subsequently regenerating a healthy hematopoietic system by transplanting HSCs from a healthy donor (allogeneic HSCs) or the patient's own healthy stem cells (autologous HSCs).

The ability to use HSCs in transplantation, however, requires the ability to isolate, expand and maintain HSCs in culture, given their very limited occurrence naturally. For example, SCT is only successful when a sufficient number of HSCs can be provided. Patient-derived peripheral blood stem-cell products can be used for autologous SCT, which supports hematopoietic rescue after high-dose chemotherapy for various types of hematological malignancies, solid tumors, and autoimmune diseases. However, in many diseases affecting blood cell lineages, the number of HSCs that can be obtained from a patient is very limited in number. Further, under in vitro cell culture conditions, the number of HSCs usually decline because HSCs either die or terminally differentiate, losing their stem cell properties.

A range of approaches have been tested to expand hematopoietic stem cells in vitro. However, stable expansion of HSCs in vitro remains a significant challenge. Techniques developed to date have been limited to the use of chemically defined culture media that play on the exposure of HSCs to cytokines, small molecules that inhibit or stimulate individual intracellular signaling pathways, and remodelers of the epigenetic state of HSCs. All these technologies have suffered from limited potency or poor reproducibility, mostly attributable to variability in purity in the different batches distributed by suppliers of these molecules.

As such, there is an urgent need for additional and improved expansion methods to expand HSCs and related cells in vitro.

### SUMMARY OF THE INVENTION

Provided herein are methods for the in vitro expansion of hematopoietic stem cells (HSCs) or hematopoietic progenitor cells (HPCs) by exposing the HSCs or HPCs to HSC-and/or HPC-derived extracellular vesicles (EVs).

Provided is a method of expanding a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

Provided is a method of increasing the expression of stem cell factor (SCF) in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

Provided is a method of increasing the capacity to reconstitute the hematopoietic system in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

Provided is a method of increasing NAD(P)H levels in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

Provided is a method of increasing the capacity for self-renewal in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

Provided is a method of lowering the mitochondrial mass in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

In some embodiments, the population of recipient cells is contacted with HSC-derived EVs and/or HPC-derived EVs at a ratio of 1-3×10\hat{ }6 (HSC-derived EVs and/or HPC-derived EVs) per (recipient HSC and/or recipient HPC).

In some embodiments, the step of substantially isolating the HSC-derived EVs and/or HPC-derived EVs from the medium is performed by ultracentrifugation, polyethylene glycol (PEG) centrifugation, or size-exclusion chromatography.

In some embodiments, the donor HSCs and/or donor HPCs are obtained from a mammal. In one embodiment, the donor HSCs and/or donor HPCs are obtained from a human.

In one embodiment, the recipient cells are obtained from a mammal. In one embodiment, the recipient cells HPCs are obtained from a human.

In one embodiment, the recipient cells are recipient HSCs. In one embodiment, the recipient cells are recipient HPCs.

In one embodiment, the donor cells are donor HSCs. In one embodiment, the donor cells are donor HPCs.

In some embodiments, the medium is based on Iscove's Modified Dulbecco's Medium (MDM), Basal Media Eagle's (BME), Dulbecco's Modified Eagle Medium (DMEM), DMEM/F-12 medium, Roswell Park Memorial Institute (RPMI) medium, RPMI 1640 medium, or Stem Span™ Serum Free Expansion Media (SFEM).

In some embodiments, the medium comprises one or more agents selected from the group consisting of thrombopoietin (TPO), stem cell factor (SCF), insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), Oct 4-activating compound 1 (OAC1), p38 MAPK inhibitor JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS).

Provided is a pharmaceutical composition comprising a population of recipient cells produced by a method disclosed herein and a pharmaceutically acceptable excipient.

Provided is a method of treating a subject in need of hematopoietic reconstitution, the method comprising administering to the subject a pharmaceutical composition disclosed herein.

Provided is a method of transplanting HSCs and/or HPCs into a subject in need thereof, the method comprising administering to the subject a pharmaceutical composition disclosed herein.

Provided is a method of treating a disease or disorder in a subject in need thereof, the method comprising administering to the subject a pharmaceutical composition disclosed herein, wherein the disease or disorder is a red blood cell disorder, anemia, congenital dyserythropoietic anemia, congenital sideroblastic anemia, G6PD deficiency, hemolytic anemia, iron-refractory iron deficiency anemia (IRIDA syndrome), pyruvate kinase (PK) deficiency, sickle cell disease, spherocytosis, thalassemia, white blood cell disorder, cyclic neutropenia, severe congenital neutropenia (Kostmann syndrome), chronic granulomatous disease, leukocyte adhesion deficiency, bone marrow failure syndromes, aplastic anemia, congenital amegakaryocytic thrombocytopenia, Diamond-Blackfan anemia, dyskeratosis congenita, Fanconi anemia, myelodysplastic syndrome (MDS), Pearson syndrome, Shwachman-Diamond syndrome, thrombocytopenia absent radius, bleeding disorder, platelet function disorder, thrombocytopenia, autoimmune blood cell disorder, autoimmune hemolytic anemia, Evans syndrome, immune thrombocytopenia (IPT), myeloproliferative neoplasm (MPN), myelofibrosis, essential thrombocytosis, polycythemia vera, polycythemia, Severe Combined Immune Deficiency Syndrome (SCID), Chediak-Higashi syndrome, Kostman syndrome, Blackfan-Diamond anemia, or Wiskott-Aldrich syndrome (WAS).

In some embodiments, the method further comprises a step of depleting the subject's bone marrow before administering to the subject a pharmaceutical composition disclosed herein. In some embodiments, the step of depleting the subject's bone marrow is performed by chemotherapy and/or radiation therapy.

Provided is a method of treating cancer in a subject in need thereof, the method comprising: (a) administering an anti-cancer treatment to the subject; and (b) administering to the subject a pharmaceutical composition disclosed herein (e.g., a pharmaceutical composition comprising a population of recipient cells produced by a method disclosed herein).

In some embodiments, the cancer is leukemia, myeloma, or lymphoma. In some embodiments, the lymphoma is Hodgkin lymphoma or non-Hodgkin lymphoma. In some embodiments, the leukemia is Acute Myeloid Leukemia (AML), Acute Lymphocytic Leukemia (ALL), Chronic Myeloid Leukemia (CML), Chronic or Lymphocytic Leukemia (CLL).

In some embodiments, the anti-cancer treatment is chemotherapy and/or radiation therapy.

Provided is a kit comprising (1) a population of recipient cells produced by a method disclosed herein or (2) a pharmaceutical composition disclosed herein.

### BRIEF DESCRIPTION OF THE FIGURES

FIG. 1 illustrates a method for expanding hematopoietic stem cells (HSCs) based on the administration of extracellular vesicles isolated from hematopoietic stem cells (HSC-EVs). Cultured donor HSCs spontaneously release EVs, which are purified. These vesicles are then administered in vitro to a separate group of HSCs (recipient HSCs). The actual expansion of recipient HSCs can be verified through in vitro assays (e.g., colony-forming unit assay) or in vivo using experimental animal models such as competitive bone marrow transplantation.

FIG. 2A. Distribution of the diameter of EVs derived from HSCs determined by nanoparticle tracking analysis (NTA). FIG. 2B. Particle size distributions of EVs derived from CD34⁻ HSCs, measured using a tunable resistive pulse sensing (TRPS) system. This technique estimates the size of nanoparticles by their capacity to interfere with the electric conductance of a membrane punctuated with nano-sized pores (pore size 100 nm) (n=3 independent experiments). CD34⁻ HSCs were cultured in the medium for 48 h to allow the release of EVs. EV-enriched media was collected and processed for EVs isolation via polyethylene glycol and centrifugation. FIG. 2C. Transmission electron microscopic images from NAD(P)H^{Hi} CD34⁻ HSC-derived EVs confirming the specific characteristics for exosomes, including a round morphology with a central depression (black arrows). Scale bar, 100 nm. EVs were isolated via polyethylene glycol and centrifugation.

FIG. 3A. Experimental strategy of EV transfer between donor and acceptor HSCs. Donor CD34⁻ HSCs (black) or compensation beads (white) were sorted into FBS-free media, and stained with the lipid marker, PKH26 (grey). EVs were isolated and supplemented to freshly isolated acceptor HSCs, then EV-related PKH26 signal (grey) was determined by fluorescence microscopy. FIG. 3B. Images (left) and quantitation (right) of PKH26 staining in acceptor CD34⁻ HSCs exposed to HSC-derived EVs (HSC-EV) or negative control (bead) (HSC-EV, n=14 cells; bead, n=11 cells).

FIG. 4. Particle size distributions of EVs from CD34⁻ HSCs, measured by TRPS system. CD34⁻ HSCs isolated from Cpt2^{f/f} Vav-iCre⁻ and Vav-iCre⁺ mice were cultured in vitro for 48 h. A fraction of cells from Cpt2^{f/f} Vav-iCre⁺ mice were exposed to cholesterol-enriched methyl-b-6 cyclodextrin (MBCD: Chol). EVs were isolated and analyzed by TRPS (n=5 independent experiments), frequency distribution of the diameter (top) and average concentration (bottom) are shown.

FIG. 5A. HSC-derived EVs were enriched with immunomagnetic beads and then stained with antibodies against EV-surface marker CD63. Quantification of CD63 positive beads in EVs obtained from CD34⁻ HSCs (from 2-month-old Cpt2^{f/f} Vav-iCre⁻ or Cpt2^{f/f} Vav-iCre⁺) exposed to MBCD: Chol to restore intracellular cholesterol levels or vehicle control (n=4 independent replicates). The same volume of isolated EVs was used for each replicate. Vav-iCre=B6N. Cg-Commd10Tg(Vav1-iCre)A2Kio/J mice. MBCD=methyl-β-cyclodextrin. Chol=cholesterol. FIG. 5B. HSC-derived EVs were enriched with immunomagnetic beads and then stained with antibodies against CD9. Quantification of CD9 positive beads in EVs obtained from CD34⁻ HSCs (from 2-month-old Cpt2^{f/f} Vav-iCre⁻ or Cpt2^{f/f} Vav-iCre⁺) exposed to MBCD:Chol to restore intracellular cholesterol levels or vehicle control (n=5 independent replicates). The same volume of isolated EVs was used for each replicate. Vav-iCre=B6N. Cg-Commd10Tg(Vav1-iCre)A2Kio/J mice. MBCD=methyl-β-cyclodextrin. Chol=cholesterol.

FIG. 6A. Experimental design and representative gating strategy used for the analysis of enhanced super-resolution radial fluctuation (eSRRF) images on HSC-derived EVs. All spots were detected using an edge-detection algorithm from the eSRRF image then measured for morphometric parameters and CD63 intensity. Spots were then gated for size, shape and intensity using the positive control (100 nm fluorescent beads) as reference. Next, the gates were adjusted to exclude spots found in the negative control (PBS) to minimize the possible contamination caused by image noise or aggregates of the fluorescent antibody. The remaining spots were considered bona fide EVs and their CD63 intensity compared across samples. FIG. 6B. The expression of EV marker CD63 at single EV level was determined by eSRRF. Images of CD63 in CD34⁻ HSCs-derived EVs (top) and quantitation of CD63 (bottom) in each (Cpt2^{f/f} Vav-iCre⁻, n=87 (condition 1); Cpt2^{f/f} Vav-iCre⁺, n=332 (condition 2); Cpt2^{f/f} Vav-iCre⁺+MBCD:Chol, n=138 (condition 3); PBS, n=10; beads, n=83 (condition 4)). FIG. 6C. Nanometric flow cytometry analysis of HSC-derived EVs immune-stained for CD63 and investigated by NanoFCM NanoAnalyzer. Traces from left to right: Conditions 2 and 4 (overlapping); condition 1, condition 3.

FIG. 7A. Quantitation of NAD(P)H autofluorescence obtained by fluorescence microscopy in CD34⁻ HSCs exposed to HSC-derived EVs (HSC-EV) or negative control (bead) (HSC-EV, n=13 cells; bead, n=11 cells). EVs were isolated via polyethylene glycol and centrifugation. FIG. 7B. Analysis of mitochondrial volume in CD34⁻ HSCs exposed to HSC-derived EVs (HSC-EV) or negative control (bead) (HSC-EV, n=13 cells; bead, n=13 cells). EVs were isolated via polyethylene glycol and centrifugation. FIG. 7C. Expression levels of SCF in HSC exposed to EVs isolated from HSC, FKSL cells, Lin cells, or MSC and obtained by qPCR relative to ActB (n=3) Ø refers to the control (no EVs). Stem cell factor (SCF) is an essential hematopoietic cytokine that interacts with other cytokines to preserve the function of hematopoietic stem and progenitor cells. The expression of SCF in HSC exposed in vitro for 48 h to HSC-derived EVs or vehicle was evaluated. While SCF expression was barely detectable in HSCs maintained in basal conditions, it significantly increased upon administration of EVs, corroborating the positive effect of HSC-derived EVs in supporting HSC function. Interestingly, EVs derived from committed progenitor (FKSL or Lin⁻) cells or MSCs could not stimulate SCF expression. FIG. 7D. Quantitation of PKH26 signal transferred to HSC from EVs isolated from HSC, FKSL cells, Lin cells, or MSC and stained with PKH26 before administration to HSC (Ø, n=499 cells; HSC, n=380 cells; FKSL, n=282 cells; Lin⁻, n=288 cells; 3 independent experiments). Ø refers to the control (no EVs). FKSL=progenitor cells. Lin⁻=more differentiated precursor cells. MSC=mesenchymal stem cells.

FIG. 8A. Experimental design (top left) and percentages of donor-derived cells (top right and bottom) in various lineages of peripheral blood of recipient mice transplanted with HSCs supplemented with HSC-derived EVs. Per each recipient mouse, twenty-five CD34⁻ HSCs were sorted and cultured for 48 h. During this period, HSCs were supplemented with HSC-derived EVs (+HSC-EV; 1.5×10⁶ EV per single HSC) (top trace in each panel). EV-free PBS was used as a vehicle (bottom trace in each panel). After 48 h of culture, the whole culture was transplanted into lethally irradiated Ly5.1 recipient mice together with 4×10⁵ competitor bone marrow mononuclear cells (BMMNCs.) FIG. 8B. Percentages of donor-derived cells in the indicate fractions of the bone marrow of the recipient mice 28 weeks after bone marrow transplantation (BMT). hematopoietic stem and progenitor cells 32 HSPCs. Left bars: +HSC-EV. Right bars: Vehicle.

FIG. 9. KSL cells were exposed to GW4869 or vehicle in the absence of stromal cells, and their serial-replating ability was determined. Colony counts (reported for each replating) were performed in independent littermate pairs (n=3). Left bars: Vehicle. Right bars: GW4869.

FIG. 10A. Experimental design (top left) and percentages of donor-derived cells (top right and bottom) in various lineages of peripheral blood of recipient mice after Rab27a interfering. After infection and selection with G418, 2,000 live FKSL cells were transplanted into lethally irradiated Ly5.1 recipient mice together with 1×10⁶ competitor BMMNCs. Doxycycline was supplemented in water starting at week 2 after BMT. The x axis represents weeks after doxycycline administration. Top trace in each panel: −Dox. Bottom trace in each panel: +Dox. FIG. 10B. Percentages of donor-derived cells in the indicated fractions of the bone marrow of the recipient mice 20 weeks after BMT. Left bars: +Dox. Right bars: −Dox.

FIG. 11A. Quantitation of PKH26 signal transferred to BM stromal cells from EVs isolated from HSC, FKSL cells, or Lin⁻ cells and stained with PKH26 before administration to BM stromal cells (Ø, n=55 cells; HSC, n=1160 cells; FKSL, n=1659 cells; Lin⁻, n=2187 cells; 3 independent experiments). Ø refers to the control (no EVs). FIG. 11B. Expression levels of SCF in BM stromal cells exposed to EVs isolated from HSC, FKSL cells, or Lin cells. Ø refers to the control (no EVs). Data were obtained by qPCR relative to ActB (Ø, n =7 replicates; HSC, n=11 replicates; FKSL, n=6 replicates; Lin⁻, n=9 replicates). FIG. 11C. Long-term culture-initiating cell (LTC-IC) capacity of CD34⁻ HSCs exposed to EV synthesis inhibitor GW4869 or vehicle (co-culture with OP-9 stromal cells, n≥12 replicates, 4 independent experiments). Upper trace: GW4869. Lower trace: Vehicle.

### DETAILED DESCRIPTION

Provided herein are methods for expanding populations of hematopoietic stem cells (HSCs) or hematopoietic progenitor cells (HPCs) using extracellular vesicles isolated from hematopoietic stem cells (HSC-derived EVs) or from HPCs (HPC-derived EVs).

### HSCs and HPCs

HSCs are multipotent cells that can develop into a large variety of blood cells, including myeloid-lineage and lymphoid-lineage cells. HSCs can both self-renew and differentiate into hematopoietic progenitor cells. HSCs can be found in several organs, including peripheral blood (PB), bone marrow (BM), and umbilical cord blood (UCB).

During hematopoiesis (i.e., the formation of blood cellular components), HSCs first diverge into HPCs. These cells then differentiate into either the common myeloid progenitor (CMP) or common lymphoid progenitor (CLP). Both CMPs and CLPs are types of oligopotent progenitor cells (progenitor cells that differentiate into only a few cell types). CMPs and CLPs continue to differentiate along cell lines into lineage-restricted progenitor cells that become final, mature blood cells. The lymphoid branch includes blood cells such as T-cells/T-lymphocytes, B-cells/B-lymphocytes, and NK-cells/Natural killer cells, which are relevant to innate and adaptive immune cells. The myeloid lineage includes blood cells such platelets, eosinophils, basophils, neutrophils, erythrocytes, granulocytes, monocytes, dendritic cells, mast cells, osteoclasts, and macrophages.

HSCs and/or HPCs can be obtained from, for example, unfractionated bone marrow, umbilical cord, and peripheral blood. In some embodiments, the HSCs and/or HPCs can be obtained from the bone marrow of experimental animals (e.g., mice, rats, cats, dogs, rabbits, guinea pigs, hamsters, sheep, pigs, horses, bovines, and non-human primates), from human bone marrow biopsy samples, or from human cord blood.

Bone marrow cells can be harvested from a donor under sterile conditions. In general, bone marrow is aspirated by needle from larger bones, including, but not limited to, the hip bone, femur, or chest bones. HSCs and/or HPCs can be mobilized and obtained from the peripheral blood. Mobilization is a process whereby the cells are stimulated out of the bone marrow space (e.g., from the hip bones and the chest bone) into the bloodstream so they are available for collection. Mozobil® (plerixafor) injection can be used for such mobilization. In some cases, Mozobil® (plerixafor) can be used in combination with granulocyte-colony stimulating factor (G-CSF) to mobilize HSCs and/or HPCs can to the peripheral blood. After collection the cells can be expanded immediately, or frozen and stored until expansion is desired.

Crude or un-fractionated blood products can be enriched for cells having HSC and/or HPCs characteristics in ways known to those of skill in the art.

HSCs and/or HPCs can be obtained from any donor. The HSCs and/or HPCs can be healthy, wild type cells. The HSCs and/or HPCs can have mutations that can adversely affect their function.

In some embodiments, the HSCs and/or HPCs are autologous to a person or patient who may later receive an expanded population of the HSCs and/or HPCs. In some embodiments, the HSCs and/or HPCs are allogeneic (i.e., derived from a different person, including, but not limited to, a healthy donor).

The HSCs and/or HPCs can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

In some embodiments, the donor HSCs and/or HPCs are from a human.

In some embodiments, the recipient HSCs and/or HPCs are from a human.

In some embodiments, the donor HSCs and/or HPCs are from a human and the recipient HSCs and/or HPCs are from a human. In some embodiments, the donor HSCs and/or HPCs are from a human and the recipient HSCs and/or HPCs are from a non-human mammal. In some embodiments, the donor HSCs and/or HPCs are from a non-human mammal and the recipient HSCs and/or HPCs are from a human.

### HSC/HPC-derived EVs

Generally speaking, extracellular vesicles (EVs) are membrane-enclosed structures of varying sizes (30-10,000 nm), which are released from cells. EVs can contain a wide range of cargo molecules, including coding and noncoding RNA, DNA, lipids, and proteins. These vesicles are capable of mediating a range of both local and distant intercellular communications via binding and/or delivery of their EV cargo to recipient cells.

EVs can be broadly classified into four subtypes, depending on their size (i.e., diameter) and method of cellular release: (1) Exosomes (30-150 nm in size), which a rise from the endosomal pathway, (2) microvesicles (50-1000 nm in size), which arise from direct plasma membrane budding, (3) large vesicles (>1000 nm), which arise from the cleavage of large cytoplasmic extensions from a (living) cell body, (4) and apoptotic bodies (>1000 nm), which arise from cytoplasmic fragmentation during programmed cell death.

As used herein, “HSC-derived EVs” are released by HSCs. HSCs producing “HSC-derived EVs” are also referred to as “donor HSCs.” As used herein, “HPC-derived EVs” are released by HPCs. HPCs producing “HPC-derived EVs” are also referred to as “donor HPCs.” “HSC-derived EVs” and “HPC-derived EVs” are collectively referred to as “HSC/HPC-derived EVs.”

Provided herein are methods of contacting HSCs and/or HPCs with HSC-derived EVs and/or HPC-derived EVs. As used herein, HSCs contacted with HSC-derived EVs and/or HPC-derived EVs are referred to as “recipient HSCs.” As used herein, HPCs contacted with HSC-derived EVs and/or HPC-derived EVs are referred to as “recipient HPCs.” Recipient HSCs and recipient HPCs are collectively referred to as “recipient cells.”

Provided herein is a method of contacting HSCs with HSC-derived EVs.

Provided herein is a method of contacting HPCs with HSC-derived EVs.

Provided herein is a method of contacting HPCs with HPC-derived EVs.

In some embodiments, the HSC-derived EVs and/or HPC-derived EVs have a diameter of about 30 nm to about 5 pm. In some embodiments, the HSC-derived EVs and/or HPC-derived EVs have a diameter of about 30 nm to about 400 nm, of about 30 nm to about 300 nm, of about 30 nm to about 200 nm, of about 40 nm to about 180 nm, of about 40 nm to about 160 nm, of about 50 nm to about 160 nm, of about 50 nm to about 150 nm, of about 50 nm to about 140 nm, or of about 50 nm to about 130 nm. In some embodiments, the HSC-derived EVs and/or HPC-derived EVs have a diameter of about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm or about 200 nm.

In some embodiments, the HSC-derived EVs and/or HPC-derived EVs present on their surface molecular marker CD63 (also referred to as granulophysin, lysosomal-associated membrane protein 3, or Melanoma-associated antigen ME491), CD9 (also referred to as dell growth-inhibiting gene 2 protein, 5H9 antigen, leukocyte antigen MIC3, tetraspanin-29 (Tspan-29), and/or CD81 (also referred to as target of the antiproliferative antibody 1(TAPA 1 ), Tetraspanin-28 (Tspan-28)).

In some embodiments, the HSC-derived EVs and/or HPC-derived EVs have a cup-shaped morphology. EV morphology may be observed, for example, by transmission electron microscopy. Methods of assessing the morphology of EVs are known to a person skilled in the art.

Generation of HSC-derived EVs and/or HPC-derived EVs

In some embodiments, HSC-derived EVs and/or HPC-derived EVs are generated by culturing isolated donor HSCs and/or donor HPCs in a suitable cell culture medium. Methods and media for culturing HSCs or HPCs are known in the art. See, e.g., Yadav et al., Hematopoietic Stem Cells Culture, Expansion and Differentiation: An Insight into Variable and Available Media, Int J Stem Cells. 2020 Nov. 30;13(3):326-334; Sakurai et al., Ex vivo expansion of human hematopoietic stem cells and clinical applications, Cancer Sci. 2024 Mar;115(3):698-705, which are incorporated herein in their entireties.

In some embodiments, the cell culture medium is derived from or based on Iscove's Modified Dulbecco's Medium (MDM), Basal Media Eagle's (BME), Dulbecco's Modified Eagle Medium (DMEM), DMEM/F-12 medium, Roswell Park Memorial Institute (RPMI) medium (also referred to as RPMI 1640 medium), or Stem Span™ Serum Free Expansion Media (SFEM) (a defined, serum-free medium consisting of IMDM, bovine serum albumin, rHuman insulin, human transferrin, 2-β-mercaptoethanol, and supplements).

In some embodiments, the cell culture medium comprises one or more of: bovine serum albumin (BSA), fetal bovine serum (FBS), insulin (e.g., recombinant human insulin), transferrin (e.g., human transferrin and/or iron-saturated transferrin), 2-mercaptoethanol, stem cell factor (SCF), thrombopoietin (TPO), Fms-like tyrosine kinase 3 ligand (FLT3L), interleukin-6, interleukin-3, interleukin-10, granulocyte-macrophage colony-stimulating factor, erythropoietin, resveratrol, glutamine, insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, laminin, caspase inhibitor, epigallocatechin gallate (EGCG), Oct4-activating compound 1 (OAC1), p38 MAPK inhibitor JAK/STAT inhibitors, human growth hormone (HGH), VEGF-C modulator or inhibitor, and ALK5/SMAD modulator or inhibitor.

In some embodiments, the cell culture medium comprises one or more of the following small molecules: methyltransferase inhibitor (e.g., UNC0638), PGE2 analog (e.g., dmPGE2), p38-13 MAPK inhibitor (e.g., C7), JNK inhibitor (e.g., JNK-IN-8), p18 inhibitor (e.g., 005A), BET inhibitor (e.g., CPI-203), inhibitor of DNA methyltransferase/HDAC (e.g., 5azaD+TSA), GSK3β inhibitor (e.g., BIO), ALDH inhibitor (e.g., DEAB), histone acetyltransferase inhibitor (e.g., garcinol), inhibitor of NAD+-dependent enzymes (e.g., Nicotinamide), inhibitor of mTOR and GSK3β (e.g., Rapamycin+CHIR99021), AhR antagonist (e.g., SR-1), Copper chelator (e.g., TEPA), HDAC inhibitor (e.g., Valproic acid), and apoptosis inhibitor (e.g., zVADfmk). See Sakurai M, Ishitsuka K, Becker HJ, Yamazaki S. Ex vivo expansion of human hematopoietic stem cells and clinical applications. Cancer Sci. 2024 Mar;115(3):698-705.

In some embodiments, the culture medium comprises SCF and/or TPO at about 50 ng/ml each. In some embodiments, the culture medium comprises SCF at a concentration of about 1 to about 60 ng/ml; at a concentration of about 2 to about 60 ng/ml; at a concentration of about 2 to about 55 ng/ml; at a concentration of about 2 to about 50 ng/ml; at a concentration of about 10 to about 50 ng/ml; at a concentration of about 20 to about 50 ng/ml; or at a concentration of about 2 to about 20 ng/ml. In one embodiment, the culture medium comprises SCF at a concentration of about 50 ng/ml. In one embodiment, the culture medium comprises SCF at a concentration of about 20 ng/ml. In some embodiments, the culture medium comprises TPO at a concentration of about 0.1 to about 60 ng/ml; at a concentration of about 1 to about 60 ng/ml; at a concentration of about 2 to about 60 ng/ml; at a concentration of about 2 to about 55 ng/ml; at a concentration of about 2 to about 50 ng/ml; at a concentration of about 10 to about 50 ng/ml; at a concentration of about 20 to about 50 ng/ml; or at a concentration of about 2 to about 20 ng/ml. In one embodiment, the culture medium comprises TPO at a concentration of about 50 ng/ml. In one embodiment, the culture medium comprises TPO at a concentration of about 20 ng/ml.

In some embodiments, the HSCs and/or HPCs are cultured at least about 24 h, at least about 36 h, at least about 48 h, at least about 72 h, or at least about 96 h before isolation of the EVs. In some embodiments, the HSCs and/or HPCs are cultured at least about 1, about 2, about 3, about 4, about 5, about 6, about 7, about 8, about 9, about 10, about 11, about 12, about 12, or about 14 days before isolation of the EVs.

### Isolation of HSC/HPC-derived EVs

HSC-derived EVs and/or HPC-derived EVs can be isolated using methods known to a person skilled in the art. In embodiments, the HSC-derived EVs and/or HPC-derived EVs are substantially purified. As used herein, “substantially purified” means that the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

In some embodiments, the HSC-derived EVs and/or HPC-derived EVs are isolated using methods and tools, including, but not limited to, ultracentrifugation (including cushion ultracentrifugation, differential ultracentrifugation, density gradient centrifugation, and rate-zonal centrifugation), filtration, polyethylene glycol (PEG) centrifugation, size-exclusion chromatography columns, density gradients, tangential flow filtration, polymer-based precipitation (suing, for example, PEG or lectin), immunoaffinity-based isolation, asymmetrical-flow field-flow fractionation, micro-, nano-fluidic chips (which isolate EVs based on their biochemical properties using acoustic, electrophoretic, and electromagnetic technology), immuno-based microfluidic isolation, or commercially available EV isolation kits. Methods of isolating EVs have been described, for example, in Zhao et al., Isolation and analysis methods of extracellular vesicles (EVs), Extracell Vesicles Circ Nucl Acids. 2021;2:80-103; Akbar et al., Methodologies to Isolate and Purify Clinical Grade Extracellular Vesicles for Medical Applications, Cells. 2022 Jan. 6;11(2):186; Veerman et al., Molecular evaluation of five different isolation methods for extracellular vesicles reveals different clinical applicability and subcellular origin, J Extracell Vesicles. 2021 Jul;10(9): e12128, all of which are incorporated herein in their entireties by reference.

In some embodiments, the HSC-derived EVs and/or HPC-derived EVs are isolated using a PEG-based method. In some embodiments, culture medium enriched with HSC-derived EVs and/or HPC-derived EVs is exposed to about 8% PEG 8000 at about 4° C. for at least about 24 h before purification. Afterwards, the EVs may be centrifuged at over 3000 g for about 1 h at about 4° C. The supernatant may be discarded, and the EV pellet may be resuspended in a suitable buffer.

The HSC-derived EVs and/or HPC-derived EVs may be stored in any suitable buffer or liquid before use. In one embodiment, the buffer is phosphate-buffered saline (PBS). Other buffers known to the person skilled in the art may be employed.

In some embodiments, HSC-derived EVs and/or HPC-derived EVs are stored at about 4° C. for up to about 24 h. In some embodiments, the HSC-derived EVs and/or HPC-derived EVs are stored at about −20° C. between about 24 h and about 7 days. In some embodiments, the HSC-derived EVs and/or HPC-derived EVs are stored at about −80° C. for longer periods.

### Methods

Provided herein are methods of contacting the population of recipient cells (recipient HSCs and/or recipient HPCs) with EVs derived from donor cells (donor HSCs and/or donor HPCs). Provided herein are methods of expanding a population of recipient cells using HSC-derived EVs and/or HPC-derived EVs.

Provided is a method of expanding a population of recipient HSCs and/or recipient HPCs, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of expanding a population of recipient HSCs, the method comprising contacting the population of recipient HSCs with HSC-derived EVs, wherein the HSC-derived EVs have been substantially isolated from a culture medium, in which a population of donor HSCs has been cultured.

Provided is a method of expanding a population of recipient HPCs, the method comprising contacting the population of recipient HPCs with HSC-derived EVs, wherein the HSC-derived EVs have been substantially isolated from a culture medium, in which a population of donor HSCs has been cultured.

Provided is a method of expanding a population of recipient HPCs, the method comprising contacting the population of recipient HPCs with HPC-derived EVs, wherein the HPC-derived EVs have been substantially isolated from a culture medium, in which a population of donor HPCs has been cultured.

Provided is a method of expanding a population of recipient HSCs or recipient HPCs, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated substantially isolated HSC-derived EVs and/or HPC-derived EVs.

Provided is a method of expanding a population of recipient HSCs, the method comprising: (a) culturing a population of donor HSCs in a medium, whereby the donor HSCs produce HSC-derived EVs; (b) substantially isolating the HSC-derived EVs from the medium, thereby obtaining substantially isolated HSC-derived EVs; and (c) contacting the population of recipient HSCs with the substantially isolated substantially isolated HSC-derived EVs.

Provided is a method of expanding a population of recipient HPCs, the method comprising: (a) culturing a population of donor HSCs in a medium, whereby the donor HSCs produce HSC-derived EVs; (b) substantially isolating the HSC-derived EVs from the medium, thereby obtaining substantially isolated HSC-derived EVs; and (c) contacting the population of recipient HPCs with the substantially isolated substantially isolated HSC-derived EVs.

Provided is a method of expanding a population of recipient HPCs, the method comprising: (a) culturing a population of donor HPCs in a medium, whereby the donor HPCs produce HPC-derived EVs; (b) substantially isolating the HPC-derived EVs from the medium, thereby obtaining substantially isolated HPC-derived EVs; and (c) contacting the population of recipient HPCs with the substantially isolated substantially isolated HPC-derived EVs.

Methods and assays for assessing the expansion of recipient HSCs and/or HPCs are known in the art. Such methods and assays include, but are not limited to, in vitro assays (e.g., colony-forming unit assay) or in vivo using experimental animal models (e.g., competitive bone marrow transplantation) (see, for example, FIG. 1). In one embodiment, human HSC activity is assessed by a mouse repopulating cell (SRC) assay using NOD-SCID or NOD. Cg-Prkdc^{scid} Il2rg^{tm1Wjl}/SzJ (NSG) mice. The number of NOD-SCID repopulating cells (SRCs) may be quantified by limiting dilution assays (LDAs).

Provided is a method of increasing the expression of stem cell factor (SCF) in a population of recipient HSCs and/or recipient HPCs, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of increasing the expression of SCF in a population of recipient HSCs, the method comprising contacting the population of recipient HSCs with HSC-derived EVs, wherein the HSC-derived EVs have been substantially isolated from a culture medium, in which a population of donor HSCs has been cultured.

Provided is a method of increasing the expression of SCF in a population of recipient HPCs, the method comprising contacting the population of recipient HPCs with HSC-derived EVs, wherein the HSC-derived EVs have been substantially isolated from a culture medium, in which a population of donor HSCs has been cultured.

Provided is a method of increasing the expression of SCF in a population of recipient HPCs, the method comprising contacting the population of recipient HPCs with HPC-derived EVs, wherein the HPC-derived EVs have been substantially isolated from a culture medium, in which a population of donor HPCs has been cultured.

Provided is a method of increasing the expression of SCF in a population of recipient HSCs or recipient HPCs, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated substantially isolated HSC-derived EVs and/or HPC-derived EVs.

Methods of assessing the expression of SCF are known in the art. In some embodiments, expression is assessed by analyzing SCF mRNA levels, for example, by using real-time PCR, Northern blotting, DNA microarrays, Serial Analysis of Gene Expression (SAGE), RNA sequencing, tiling arrays, or other methods known in the art. In some embodiments, expression is assessed by analyzing SCF protein levels, for example, by Western blotting, immunoassays, mass spectrometry, or other methods known in the art.

Provided is a method of increasing in a population of recipient HSCs and/or recipient HPCs the capacity to reconstitute the hematopoietic system, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of increasing in a population of recipient HSCs the capacity to reconstitute the hematopoietic system, the method comprising contacting the population of recipient HSCs with HSC-derived EVs, wherein the HSC-derived EVs have been substantially isolated from a culture medium, in which a population of donor HSCs has been cultured.

Provided is a method of increasing in a population of recipient HPCs the capacity to reconstitute the hematopoietic system, the method comprising contacting the population of recipient HPCs with HSC-derived EVs, wherein the HSC-derived EVs have been substantially isolated from a culture medium, in which a population of donor HSCs has been cultured.

Provided is a method of increasing in a population of recipient HPCs the capacity to reconstitute the hematopoietic system, the method comprising contacting the population of recipient HPCs with HPC-derived EVs, wherein the HPC-derived EVs have been substantially isolated from a culture medium, in which a population of donor HPCs has been cultured.

Provided is a method of increasing in a population of recipient HPCs or recipient HPCs the capacity to reconstitute the hematopoietic system, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated HSC-derived EVs and/or HPC-derived EVs.

Methods of assessing the ability of cells to reconstitute the hematopoietic system are known in the art. See, e.g., Szilvassy et al., Quantitative assay for totipotent reconstituting hematopoietic stem cells by a competitive repopulation strategy, Proc Natl Acad Sci U S A. 1990 Nov;87(22):8736-40; van Os et al., Stem cell assays: something old, something new, something borrowed, Stem Cells. 2004;22(7):1181-90, which are incorporated herein by reference in their entireties.

Provided is a method of increasing NAD(P)H levels in a population of recipient HSCs and/or recipient HPCs, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of increasing NAD(P)H levels in a population of recipient HSCs, the method comprising contacting the population of recipient HSCs with HSC-derived EVs, wherein the HSC-derived EVs have been substantially isolated from a culture medium, in which a population of donor HSCs has been cultured.

Provided is a method of increasing NAD(P)H levels in a population of HPCs, the method comprising contacting the population of recipient HPCs with HSC-derived EVs, wherein the HSC-derived EVs have been substantially isolated from a culture medium, in which a population of donor HSCs has been cultured.

Provided is a method of increasing NAD(P)H levels in a population of HPCs, the method comprising contacting the population of recipient HPCs with HPC-derived EVs, wherein the HPC-derived EVs have been substantially isolated from a culture medium, in which a population of donor HPCs has been cultured.

Provided is a method of increasing NAD(P)H levels in a population of recipient HPCs or recipient HPCs, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated substantially isolated HSC-derived EVs and/or HPC-derived EVs.

Methods of assessing NAD(P)H levels are known in the art. See, e.g., Azouaoui et al., Meta-analysis of NAD(P)(H) quantification results exhibits variability across mammalian tissues, Sci Rep. 2023 Feb 11;13(1):2464, which is incorporated herein in its entirety.

Provided is a method of increasing the capacity for self-renewal in a population of recipient HSCs and/or recipient HPCs, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of increasing the capacity for self-renewal in a population of recipient HSCs, the method comprising contacting the population of recipient HSCs with HSC-derived EVs, wherein the HSC-derived EVs have been substantially isolated from a culture medium, in which a population of donor HSCs has been cultured.

Provided is a method of increasing the capacity for self-renewal in a population of HPCs, the method comprising contacting the population of recipient HPCs with HSC-derived EVs, wherein the HSC-derived EVs have been substantially isolated from a culture medium, in which a population of donor HSCs has been cultured.

Provided is a method of increasing the capacity for self-renewal in a population of HPCs, the method comprising contacting the population of recipient HPCs with HPC-derived EVs, wherein the HPC-derived EVs have been substantially isolated from a culture medium, in which a population of donor HPCs has been cultured.

Provided is a method of increasing the capacity for self-renewal in a population of recipient HPCs or recipient HPCs, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated substantially isolated HSC-derived EVs and/or HPC-derived EVs.

Methods for assessing the capacity for self-renewal are known in the art and include any methods describe herein, including the methods in Example 5.

Provided is a method of increasing the mitochondrial mass in a population of recipient HSCs and/or recipient HPCs, the method comprising contacting the population of recipient HSCs and/or recipient HPCs with HSC-derived EVs and/or HPC-derived EVs, wherein the HSC-derived EVs and/or HPC-derived EVs have been substantially isolated from a culture medium, in which a population of donor HSCs and/or donor HPCs has been cultured.

Provided is a method of increasing the mitochondrial mass in a population of recipient HSCs, the method comprising contacting the population of recipient HSCs with HSC-derived EVs, wherein the HSC-derived EVs have been substantially isolated from a culture medium, in which a population of donor HSCs has been cultured.

Provided is a method of increasing the mitochondrial mass in a population of HPCs, the method comprising contacting the population of recipient HPCs with HSC-derived EVs, wherein the HSC-derived EVs have been substantially isolated from a culture medium, in which a population of donor HSCs has been cultured.

Provided is a method of increasing the mitochondrial mass in a population of HPCs, the method comprising contacting the population of recipient HPCs with HPC-derived EVs, wherein the HPC-derived EVs have been substantially isolated from a culture medium, in which a population of donor HPCs has been cultured.

Provided is a method of increasing the increasing the mitochondrial mass in a population of recipient HPCs or recipient HPCs, the method comprising: (a) culturing a population of donor HSCs and/or donor HPCs in a medium, whereby the donor HSCs produce HSC-derived EVs and/or the donor HPCs produce HPC-derived EVs; (b) substantially isolating the HSC-derived EVs and/or the HPC-derived EVs from the medium, thereby obtaining substantially isolated HSC-derived EVs and/or HPC-derived EVs; and (c) contacting the population of recipient HSCs and/or HPCs with the substantially isolated substantially isolated HSC-derived EVs and/or HPC-derived EVs.

Methods for assessing mitochondrial mass are known in the art. For example, the mitochondrial mass can be determined by measuring the mtDNA: nDNA ratio in PBMCs using quantitative real-time PCR (qRT-PCR). Alternatively, mitochondrial proteins (including, but not limited to, voltage-dependent anion-selective channel protein (VDAC), superoxide dismutase [Mn] (MnSOD), electron transport chain (ETC) subunits) may be normalized to cytosolic proteins (e.g. actin).

When reference is made to an “increase” or a “decrease,” for example, in the context of expression levels of SCF, the capacity to reconstitute the hematopoietic system, NAD(P)H levels, the capacity for self-renewal, or mitochondrial mass, the increased or decreased level in question may be compared to a reference or control level. The reference or control level may refer to an absolute value, e.g., a concentration, a ratio of concentrations, a ratio of amounts, etc. The reference or control level may be derived from a control population. The reference or control level may be derived from a cell population that has not been contacted by HSC-derived EVs and/or HPC-derived EVs. The reference or control level may be derived from a cell population before the cell population has been contacted by HSC-derived EVs and/or HPC-derived EVs. An increase or decrease may refer to an increase or decrease over time.

In some embodiments, provided is a method of culturing HSCs and/or HPCs with HSC-derived EVs and/or HPC-derived EVs, wherein HSC-derived EVs and/or HPC-derived EVs are added to the culturing medium multiple times during a cultivation period. For example, in one embodiment, HSC-derived EVs and/or HPC-derived EVs are added to a culture medium comprising recipient cells and the recipient cells are cultured in presence of the HSC-derived EVs and/or HPC-derived EVs. After a certain period of time, e.g., after about 12 h, about 24 h, about 36 h, about 48 h, about 60 h, or about 72 h, a second bolus of HSC-derived EVs and/or HPC-derived EVs is added to the recipient cells in culture. A third, fourth, fifth, sixth, and so on bolus may be added. In some embodiments, HSC-derived EVs and/or HPC-derived EVs are added to the recipient cells daily or every other day.

In some embodiments, the contacting step comprises contacting the recipient cells with about 1-3×10⁶ EVs per HSC or HPC. In one embodiment, the contacting step comprises contacting the recipient cells with about 1.5×10⁶ EVs per HSC or HPC.

The recipient cells are usually cultured in a suitable cell culture medium. Methods and media for culturing HSCs or HPCs are known in the art. See, e.g., Yadav et al., Hematopoietic Stem Cells Culture, Expansion and Differentiation: An Insight into Variable and Available Media, Int J Stem Cells. 2020 Nov 30;13(3):326-334; Sakurai et al., Ex vivo expansion of human hematopoietic stem cells and clinical applications, Cancer Sci. 2024 Mar;115(3):698-705, which are incorporated herein by reference in their entireties.

In some embodiments, the cell culture medium is derived from or based on MDM, BME, DMEM, DMEM/F-12 medium, RPMI, or SFEM.

In some embodiments, the cell culture medium comprises one or more of: BSA, FBS, insulin (e.g., recombinant human insulin), transferrin (e.g., human transferrin and/or iron-saturated transferrin), 2-mercaptoethanol, SCF, TPO, Fms-FLT3L, interleukin-6, interleukin-3, interleukin-23 10, granulocyte-macrophage colony-stimulating factor, erythropoietin, resveratrol, glutamine, IGF-1, EDF, HGF, EGF, HSF, PTN, bFGF, ANG1, VEGF165, laminin, caspase inhibitor, EGCG, OAC1, p38 MAPK inhibitor JAK/STAT inhibitors, human growth hormone (HGH), VEGF-C modulator or inhibitor, and ALK5/SMAD modulator or inhibitor.

In some embodiments, the cell culture medium comprises one or more of the following small molecules: methyltransferase inhibitor (e.g., UNC0638), PGE2 analog (e.g., dmPGE2), p38-MAPK inhibitor (e.g., C7), JNK inhibitor (e.g., JNK-IN-8), p18 inhibitor (e.g., 005A), BET inhibitor (e.g., CPI-203), inhibitor of DNA methyltransferase/HDAC (e.g., 5azaD+TSA), GSK3β inhibitor (e.g., BIO), ALDH inhibitor (e.g., DEAB), histone acetyltransferase inhibitor (e.g., garcinol), inhibitor of NAD+-dependent enzymes (e.g., Nicotinamide), inhibitor of mTOR and GSK3β (e.g., Rapamycin+CHIR99021), AhR antagonist (e.g., SR-1), Copper chelator (e.g., TEPA), HDAC inhibitor (e.g., Valproic acid), and apoptosis inhibitor (e.g., zVADfmk). See Sakurai M, Ishitsuka K, Becker H J, Yamazaki S. Ex vivo expansion of human hematopoietic stem cells and clinical applications. Cancer Sci. 2024 Mar;115(3):698-705.

In some embodiments, the culture medium comprises SCF and/or TPO at 50 ng/ml each. In some embodiments, the culture medium comprises SCF at a concentration of about 1 to about 60 ng/ml; at a concentration of about 2 to about 60 ng/ml; at a concentration of about 2 to about 55 ng/ml; at a concentration of about 2 to about 50 ng/ml; at a concentration of about 10 to about 50 ng/ml; at a concentration of about 20 to about 50 ng/ml; or at a concentration of about 2 to about 20 ng/ml. In one embodiment, the culture medium comprises SCF at a concentration of about 50 ng/ml. In one embodiment, the culture medium comprises SCF at a concentration of about 20 ng/ml. In some embodiments, the culture medium comprises TPO at a concentration of about 0.1 to about 60 ng/ml; at a concentration of about 1 to about 60 ng/ml; at a concentration of about 2 to about 60 ng/ml; at a concentration of about 2 to about 55 ng/ml; at a concentration of about 2 to about 50 ng/ml; at a concentration of about 10 to about 50 ng/ml; at a concentration of about 20 to about 50 ng/ml; or at a concentration of about 2 to about 20 ng/ml. In one embodiment, the culture medium comprises TPO at a concentration of about 50 ng/ml. In one embodiment, the culture medium comprises TPO at a concentration of about 20 ng/ml.

In some embodiments, the HSCs and/or HPCs are cultured at least about 24 h, at least about 36 h, at least about 48 h, at least about 72 h, or at least about 96 h after addition of a bolus of EVs. In some embodiments, the HSCs and/or HPCs are cultured at least about 1, about 2, about 3, about 4, about 5, about 6, about 7, about 8, about 9, about 10, about 11, about 12, about 12, or about 14 days after addition of a bolus of EVs.

The recipient cells may be further purified, concentrated, or enriched after culturing/expanding before further processing. Methods of cell purification, concentration, or enrichment are known to a person skilled in the art.

Provided herein are methods of treating a disorder or disease in a subject in need thereof using the compositions disclosed herein. Provided herein are methods of treating a disorder or disease in a subject in need thereof using HSCs and/or HPCs produced by and/or subjected to the methods disclosed herein. For the avoidance of doubt, any of the HSCs and/or HPCs produced by and/or subjected to the methods disclosed herein are HSCs and/or HPCs disclosed herein.

As used herein, the terms “subject” and “patient” are used interchangeably irrespective of whether the subject has undergone treatment in the past or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, including, but not limited to, a cynomolgus monkey, chimpanzee, etc.) and a human). The subject may be a human or a non-human. In some embodiments, the subject is a human. In some embodiments, the subject is immune-depleted.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The terms “prevent”, “prevention”, and the like refer to acting prior to overt disease or disorder onset, to prevent the disease or disorder from developing or to minimize the extent of the disease or disorder or slow its course of development.

The compositions (including pharmaceutical compositions) disclosed herein may be administered in therapeutically effective amounts. An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

Populations or HSCs and/or HPCs can be administered to the patient in form of a pharmaceutical composition.

Provided is a method of treating a disease or disorder in a subject in need thereof, the method comprising administering to the subject a population of HSCs and/or HPCs disclosed herein (or a pharmaceutical composition comprising a population of HSCs and/or HPCs disclosed herein). Provided herein is the use of a population of HSCs and/or HPCs disclosed herein (or a pharmaceutical composition comprising a population of HSCs and/or HPCs disclosed herein) for treating a disease or disorder in a subject in need thereof. Provided herein is the use of a population of HSCs and/or HPCs disclosed herein in the manufacture of a medicament for treating a disease or disorder in a subject in need thereof. In embodiments, the disease or disorder is a red blood cell disorder, anemia, congenital dyserythropoietic anemia, congenital sideroblastic anemia, G6PD deficiency, hemolytic anemia, iron-refractory iron deficiency anemia (IRIDA syndrome), pyruvate kinase (PK) deficiency, sickle cell disease, spherocytosis, thalassemia, white blood cell disorder, cyclic neutropenia, severe congenital neutropenia (Kostmann syndrome), chronic granulomatous disease, leukocyte adhesion deficiency, bone marrow failure syndromes, aplastic anemia, congenital amegakaryocytic thrombocytopenia, Diamond-Blackfan anemia, dyskeratosis congenita, Fanconi anemia, myelodysplastic syndrome (MDS), Pearson syndrome, Shwachman-Diamond syndrome, thrombocytopenia absent radius, bleeding disorder, platelet function disorder, thrombocytopenia, autoimmune blood cell disorder, autoimmune hemolytic anemia, Evans syndrome, immune thrombocytopenia (IPT), myeloproliferative neoplasm (MPN), myelofibrosis, essential thrombocytosis, polycythemia vera, polycythemia, Severe Combined Immune Deficiency Syndrome (SCID), Chediak-Higashi syndrome, Kostman syndrome, Blackfan-Diamond anemia, or Wiskott-Aldrich syndrome (WAS). A patient might have mor one disease or disorder.

In embodiments, the population of HSCs and/or HPCs disclosed herein (or the pharmaceutical compositions comprising a population of HSCs and/or HPCs disclosed herein) is used in a stem cell transplantation (SCT)-based therapy. In embodiments, the population of HSCs and/or HPCs disclosed herein (or the pharmaceutical compositions comprising a population of HSCs and/or HPCs disclosed herein) can be used for hematopoietic rescue after high-dose chemotherapy for various types of hematological malignancies, solid tumors, and autoimmune diseases.

In some embodiments, the methods provided herein further comprise a step of depleting the patient's bone marrow before administering to the subject a population of HSCs and/or HPCs disclosed herein (or a pharmaceutical compositions comprising a population of HSCs and/or HPCs disclosed herein). In embodiments, the patient's bone marrow depletion is performed by chemotherapy and/or radiation therapy.

Provided is a method of treating cancer in a subject in need thereof, the method comprising: (a) administering an anti-cancer treatment to the subject; and (b) administering to the subject a population of HSCs and/or HPCs disclosed herein (or a pharmaceutical composition comprising a population of HSCs and/or HPCs disclosed herein). In embodiments, the cancer is leukemia, myeloma, or lymphoma. In embodiments, the lymphoma is Hodgkin lymphoma or non-Hodgkin lymphoma.

In some embodiments, the method further includes administering to the subject a second therapeutic agent. In some embodiments, the composition is administered to the subject before, after, or concurrently with the second therapeutic agent.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. The HSCs and/or HPCs disclosed herein, the pharmaceutical compositions comprising a population of HSCs and/or HPCs disclosed herein, and/or the methods described herein may be used as part of a combination therapy.

In some embodiments, the second therapeutic agent is an anti-cancer or anti-tumor agent.

In some embodiments, the method further comprises administering a therapeutically effective amount of an immune checkpoint modulator. Checkpoint proteins interact with specific ligands that send a signal into the T cell and switch off or inhibit T cell function. By expressing high levels of checkpoint proteins on their surface, cancer cells can control the function of T cells that enter the tumor microenvironment, thus suppressing the anticancer immune response. Examples of immune checkpoint modulators include PD1, PDL1, CTLA4, TIM3, LAG3, and TRAIL. The immune checkpoint protein Programmed Death-1 (PD-1) is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction can promote potent antitumor activity. Examples of PD-1 inhibitors include, but are not limited to, Pembrolizumab (MK-3475), Nivolumab (MDX-1106), Cemiplimab-rwlc (REGN2810), Pidilizumab (CT-011), Spartalizumab (PDR001), tislelizumab (BGB-A317), PF-06801591, AK105, BCD-100, BI 754091, JS001, LZM009, MEDI0680, MGA012, Sym021, TSR-042. Examples of PD-L1 inhibitors include, but are not limited to, Atezolizumab (MPDL3280A), Durvalumab (MEDI4736), Avelumab (MSB0010718C), BGB-A333, CK-301, CS1001, FAZ053, KN035, MDX-1105, MSB2311, SHR-1316. The checkpoint modulators may be administered simultaneously, separately, or concurrently with the compositions disclosed herein.

In some embodiments, the method further comprises administering a therapeutically effective amount of a “chemotherapeutic agent,” which is a chemical compound useful in the treatment of cancer. Examples of chemotherapeutic agents include alkylating agents including, but not limited to, thiotepa and cyclophosphamide; alkyl sulfonates including, but not limited to, busulfan, improsulfan and piposulfan; aziridines including, but not limited to, benzodopa, carboquone, methyldopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards including, but not limited to, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas including, but not limited to, carmustine, chlorozotocin, fotemustine, lomustine, nimustine, ranimustine; antibiotics including, but not limited to, the enediyne antibiotics (e.g. calicheamicin); dynemicin, including dynemicin A; an esperamicin; as well as neocarzinostatin chromophore and related chromoprotein enediyne antibiotics chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites including, but not limited to, methotrexate and 5-fluorouracil (5-FU); folic acid analogues including, but not limited to, denopterin, methotrexate, pteropterin, trimetrexate; purine analogs including, but not limited to, fludarabine, 6-mercaptopurine, thiamiprine, thioguanine; pyrimidine analogs including, but not limited to, ancitabine, azacitidine, 6-azauridine, carmofur, cytarabine, dideoxyuridine, doxifluridine, enocitabine, floxuridine, 5-FU; androgens including, but not limited to, calusterone, dromostanolone propionate, epitiostanol, mepitiostane, testolactone; anti-adrenals including, but not limited to, aminoglutethimide, mitotane, trilostane; folic acid replenisher including, but not limited to, frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids including, but not limited to, maytansine and ansamitocins; mitoguazone; mitoxantrone; mopidamol; nitracrine; pentostatin; phenamet; pirarubicin; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs including, but not limited to, cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors including, but not limited to, anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens including, but not limited to, flutamide, nilutamide, bicalutamide, leuprolide, xeloda, gemcitabine, KRAS mutation covalent inhibitors and goserelin; and pharmaceutically acceptable salts, acids or derivatives of any of the above. Additional examples include irinotecan, oxaliplatinum, and other standard colon cancer regimens.

### Pharmaceutical Compositions

In one embodiment, provided herein is a pharmaceutical composition comprising a population of HSCs and/or HPCs produced by and/or subjected to the methods disclosed herein and a pharmaceutically acceptable carrier.

Pharmaceutically-acceptable carriers are determined in part by the particular composition being administered, as well as by the particular method used to administer the composition. The pharmaceutical compositions may generally be formulated in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the compositions disclosed herein, use of the media or compound in the compositions disclosed herein is contemplated. In some embodiments, a second therapeutic agent can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or PBS. The composition may be sterile and fluid to the extent that easy syringeability exists. In embodiments, the compositions disclosed herein are stable under the conditions of manufacture and storage and preserved against the contaminating action of microorganisms including, but not limited to, bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating including, but not limited to, lecithin, by the maintenance of the required particle size in the case of dispersion and by the use of surfactants.

In some embodiments, the pharmaceutical composition further includes a cryo-protectant (e.g., glycerol, DMSO, PEG).

### Kits

Provided herein is a kit comprising HSCs and/or HPCs produced by and/or subjected to the methods disclosed herein. Provided herein is a kit comprising HSC-derived EVs and/or HPC-derived EVs.

The kit may include additional agents, including any agents disclosed herein.

The kit can also include a suitable container, for example, vials, tubes, mini-or microfuge tubes, test tube, flask, bottle, syringe or other container. Where an additional component or agent is provided, the kit can contain one or more additional containers into which this agent or component may be placed. Kits herein will also typically include a means for containing the HSCs, HPCs, HSC-derived EVs, and/or HPC-derived EVs and any other reagent containers in close confinement for commercial sale. Such containers may include injection or blow-molded plastic containers into which the desired vials are retained.

The kits disclosed herein may, for example, be used for expanding donor-derived HSCs and/or HPCs in vitro before transplantation into the recipient subject. The kits disclosed herein are also useful for preclinical and clinical research, e.g., with a focus on studying and utilizing HSCs and HPCs.

It is to be understood that this disclosure is not limited to the particular molecules, compositions, methodologies, or protocols described, as these may vary. Any methods and materials similar or equivalent to those described herein can be used in the practice or testing of embodiments disclosed herein. It is further to be understood that this disclosure includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments disclosed herein.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced publications, patents and applications are incorporated herein by reference in their entireties. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present disclosure, the following examples of specific embodiments are given. The following examples should not be read to limit or define the entire scope of the disclosure.

### EXAMPLES

### Example 1: Generation and characteristics of HSC-derived EVs

HSCP culture conditions

Sorted HSC or CD34⁻ Lin⁻Sca-1⁺c-Kit⁺ (KSL) were cultured in StemSpan™ SFEM (STEMCELL Technologies) supplemented with stem cell factor (SCF, PeproTech) and thrombopoietin (TPO, PeproTech) at 50 ng/ml unless otherwise described. According to the experimental need, the media could be supplemented with one of the following chemicals: Etomoxir 10 mM (an irreversible inhibitor of carnitine palmitoyltransferase 1a (CPT-1a), Merk), Lovastatin 37.5 nM (a HMG-COA reductase inhibitor, United States Pharmacopoea), GW4869 (N,N′-Bis[4-(4,5-dihydro-1H-imidazol-2-yl)phenyl]-3,3′-p-phenylene-bis-acrylamide dihydrochloride) 10 mM (inhibits exosome generation, Merk), GW501516 (2-[2-methyl-4-[[4-methyl-2-[4-(trifluoromethyl)phenyl]-1,3-thiazol-5-yl]methylsulfanyl]phenoxy]acetic acid) 0.1 mM (a PPARβ agonist, Santa Cruz Biotechnology), BIBB 515 (1-(4-chlorobenzoyl)-4-[[4-(4,5-dihydro-2-oxazolyl)phenyl]methylene]-piperidine) 12.5 mM (an inhibitor of 2,3 Oxidosqualene cyclase, Cayman Chemical), Trimethyl 1,3,5-benzenetricarboxylate (BTC) 5 mM (Merk, #Cat 115983), BMS-303141(3,5 -Dichloro-2-hydroxy-N-(4-methoxy[1,1′-biphenyl]-3-yl)-benzenesulfonamide) 1 mM (ATP-citrate lyase (ACL) inhibitor, Merk), Methyl-β-cyclodextrin (MBCD) 1.5 mM (Sigma-Aldrich).

### EVs Isolation

3000 HSC or FKSL (Lin⁻Sca-1⁺c-Kit⁺Flat3⁻) or Lin⁻ Nestin⁺ mesenchymal stem cells were sorted and cultured in StemSpan™ SFEM (STEMCELL Technologies) supplemented with 50 ng/mL stem cell factor (PeproTech) and 50 ng/mL thrombopoietin (PeproTech) for 48 h. EVs were isolated by a modified PEG based method. Specifically, EV-enriched media were exposed to 8% PEG 8000 (Merk) at 4° C. for 24 h, then EVs were centrifuged at 3214 g for 1 h at 4° C. The supernatant was discarded and the EV pellet resuspended in 30 mL of particle-free PBS.

### Nanoparticle Tracking Analysis

Nanoparticle tracking analysis (NTA) uses laser light scattering and Brownian motion to determine EVs size and concentration. This allows determining the size of particles in a suspension that flows through a focused laser beam. The light scattered by individual particles is recorded over time to visualize and track their movement. The mean square displacement for each particle along its track allows to calculate individual particle sizes using the Stokes-Einstein equation. Measurement of particle size and particle size distribution was performed with Nanosight NS300 (Malvern) instrument equipped with a 488 nm laser. All samples were diluted in filtered PBS to a final volume of 1 mL. Ideal measurement concentrations were found by pre-testing the ideal particle per frame value (20-100 particles/frame). For each measurement, four to five 1-min videos were captured under temperature 25° C. and syringe pump speed 30. Data are represented as averaged finite track length adjustment (FTLA) concentration/size.

### Tunable Resistive Pulse Sensing (TRPS)

EVs concentration and size distribution were assessed using a qNano instrument (Izon Science) equipped with an NP 150 nanopore (70-420 nm). 35 mL of each sample were loaded in the upper fluid cell, and voltage, pressure and stretch of the system were adjusted accordingly. TRPS (Tunable resistive pulse sensing) measurements as well as data analysis were performed with the Izon Control Suite Software (V3.4).

Transmission Electron Microscopy For mitochondrial analysis, 5000 sorted stem and progenitor cells were fixed in 2.5% glutaraldehyde, 2% paraformaldehyde in sodium cacodylate buffer 0.1 M, pH 7.4 for 3 h at 4° C. Samples were post-fixed in 1.0% aqueous osmium tetroxide (pH 7.4) followed by 1% uranyl acetate, dehydrated in a graded series of ethanol, and embedded in Lx112 resin (LADD Research Industries). Ultrathin (80 nm) sections were cut on a Leica UC7, contrasted with uranyl acetate followed by lead citrate, and viewed on a JEOL 1200EX transmission electron microscope (Jeol Ltd.) at 80 kV. Images were manually segmented by the TrakEM 2 plugin available on Fiji. For isolated EVs. EVs were deposited on carbonated grids, fixed in 2% PFA and stained with a contrasting solution of methyl-cellulose. Samples were then imaged with a TEM Zeiss EM 910 microscope (Zeiss).

### Results

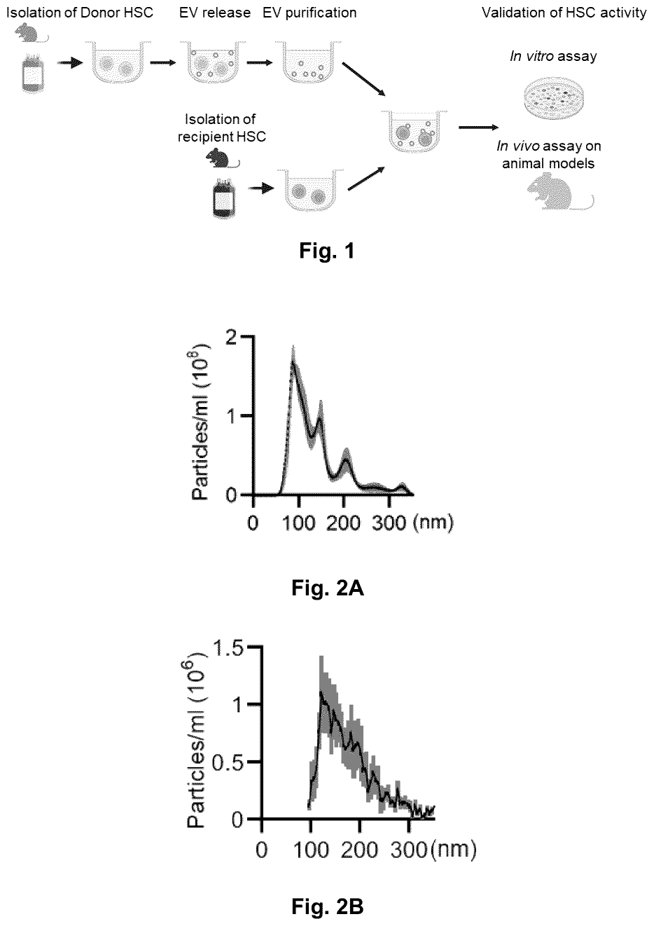

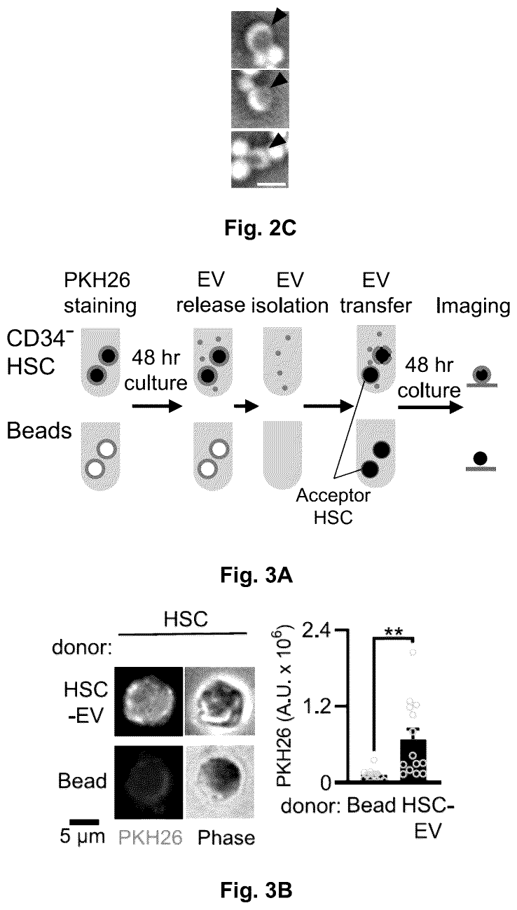

HSCs were cultured in fetal bovine serum (FBS)-free media to allow the enrichment in the media of any spontaneously released EVs. Nanoparticle tracking analysis (NTA) identified a significant amount of EVs released by HSCs, with an average diameter of ˜95 nm (FIG. 2A) confirmed by a tunable resistive pulse-sensing (TRPS) system (median diameter of ˜112 nm) (FIG. 2B). Finally, transmission electron microscopy was used to confirm that EVs from HSCs exhibit a cup-like morphology typical of exosomes (FIG. 2C).

### Example 2: HSCs Can Produce Transferable HSC-derived EVs

### EV Transfer

Sorted CD34⁻ HSC were stained with 2 mM PKH26 (Sigma-Aldrich) for 2 min at room temperature, then washed three times with medium, here Stem Span™ SFEM. PKH26-stained cells were plated in a 96 well plate and cultured in StemSpan™ SFEM medium as described herein. After 48 h, the media was collected and EVs isolated as described herein. Isolated EVs were administered to freshly isolated CD34⁻ HSC every 24 h for 48 h.

### Results

To test whether HSCs could produce transferable EVs, PKH26 was used. This lipid-specific fluorescent marker incorporates with double-layered membranes and is retained by EVs after their release (FIG. 3A, top). As a negative control, the same number of compensation beads (with no EV release) were processed in parallel (FIG. 3A, bottom). HSCs exposed to PKH26-stained EVs only displayed a marked PKH26 signal at the cell edge and in the cytosol (FIG. 3B). This data shows that HSCs can produce transferable HSC-derived EVs.

### Example 3: Impact of Defects in the NADPH-cholesterol Axis on HSC-derived EV Biogenesis

Investigation of HSC function has revealed its dependence on mitochondrial fatty acid oxidation (FAO)—the catabolismof fatty acids in the mitochondrial matrix that feeds the TCA cycle. Rate-limiting steps in FAO are represented by carnitine O-palmitoyltransferases 1 and 2 (Cpt1 and Cpt2).

To determine the impact of defects in the NADPH-cholesterol axis on EV biogenesis, CD34⁻ HSCs were isolated from Cpt2^{f/f} Vav-iCre⁻ or Vav-iCre⁺ mice and cultured for 48 h.

The conditional deletion of Cpt2 significantly affected the number of EVs retrieved, but this was partially recovered by exposure of HSCs to MBCD: Chol (FIG. 4). The diameter of these EVs was not affected (data not shown).

### Example 4: Expression of CD63 and CD9 on HSC-derived EVs Derived from Cpt2-deleted HSCs

### EV Immunomagnetic Isolation and Flow Cytometry

35 mL of EVs suspension was incubated with CD63 magnetic beads (Thermo Fisher Scientific). Isolated CD63-positive EVs were then labeled with an anti-CD63 monoclonal antibody (PE mouse anti-human CD63; eBioscience, Cat# 12-0639-42) or anti-CD9 monoclonal antibody (PerCP/Cyanine 5.5 anti-human CD 9, BioLegend, Cat# 312109). Negative control was performed by staining PBS (vehicle) instead of EVs. Flow cytometric experiments were conducted with Attune NxT Acoustic Focusing Cytometer (Life Technologies), and data were analyzed using FlowJo 10 (Becton Dickinson).

### Super-Resolution Radial Fluctuation Imaging of EVs Markers

Isolated EVs were seeded on a coverslip cleaned by acid wash and coated with poly-lysine for 60 min at 37 C to allow adhesion. EVs were then fixed with filtered 2% PFA for 10 min then blocked with filtered 5% BSA for 60 min at room temperature. Anti-CD 63 (Abcam, Cat# ab 134045) antibody was diluted 1:100 in filtered 5% BSA and incubated overnight at 4° C. After primary antibody hybridization, samples were washed three time with filtered PBS, then incubated with secondary antibodies goat anti-rabbit conjugated with Alexa 488 (Thermo Fisher Scientific). A coverslip containing fluorescent beads (TetraSpeck, Thermo Fisher Scientific) or PBS only were used as a positive and negative references for EVs detection.

Imaging was performed using a Nikon ECLIPSE Ti inverted widefield microscope equipped with 603 objective (CFI Plan Apo Lambda 60 x/1.4 oil) and an additional 1.53 magnification was used to collect fluorescence onto an EMCCD camera (iXonEM+ 885, Andor), yielding a pixel size of 88 nm. Excitation was provided by a Lambda LS Stand-Alone 175W Xenon Arc Lamp filtered through a FITC excitation filter (FF01-494/20, Semrock) and a neutral density filter (ND8, Nikon). For each field of view was acquired a 200 frames stack with exposure of 164 ms. Images were processed through the eSRRF plugin on ImageJ. Before processing the whole dataset, parameter sweep function was used then radius value was set at 4, sensitivity was set to 4. The processed eSRRF images were then analyzed using Olympus scanR. Briefly, spots were identified in all images using the edge detection function. For each spot, area, circularity and CD63 total intensity were collected. The gating strategy was also applied for analysis using Olympus scanR.

nFCM Analysis

For each staining reaction, 2×10⁸−2×10⁹ of purified EVs were incubated with CD63-APC (a 15712, life technologies) primary antibodies for 1 h at 37° C. under shaking, protected from light. Unbound antibodies were removed by repeated washing (6-8 times) with 500 mL PBS prior to analysis. Samples were next acquired using the NanoAnalyzer (nanoFCM Inc.).

Before each experiment, the NanoAnalyzer was aligned using polystyrene QC beads (nanoFCM Inc.). Size and concentration standard nanospheres (nanoFCM Inc.) were read directly after to calibrate the instrument for EV analysis. Once the nFCM was aligned and calibrated, EV samples were diluted in filtered PBS (blank) to the optimal range for measurement (108 particles/mL). Samples and blanks (200-800 events) were measured for 1 min, applying a laser power of 15 mW as excitation source, constant pressure of 1 kPa, and at an event rate between 2500 and 12,000 events/min (as recommended by manufacturers). SSC was set as the trigger channel, each particle that generated a signal above the SSC threshold was acquired as an event. For each event that also generated a signal above thresholds set in the fluorescent channels, the fluorescence intensity was registered. Empty staining reactions (without EVs) were performed as a control for all fluorescent reagents and measured under the same conditions as complete reactions (with EVs). Data analysis was performed using FlowJo 10 (Becton Dickinson).

### Results

Tetraspanins are a family of transmembrane proteins found in EVs that participate in their biogenesis and cargo selection. Cholesterol metabolism supports the cycling of tetraspanins (including CD9 and CD63) within the endomembrane system, and inhibition of cholesterol biosynthesis impairs tetraspanin loading into EVs. Thus, the expression of CD63 and CD9 on EVs derived from Cpt2-deleted HSCs was investigated using a flow cytometry and immunomagnetic beads-based methodology.

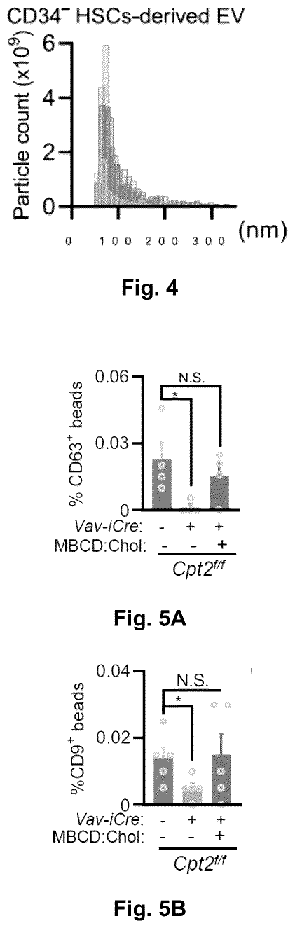

Genetic inactivation of FAO resulted in the drastic reduction of CD63 and CD9 in EVs from HSCs (FIGS. 5A and 5B), and these defects can be reverted by the replenishment of intracellular cholesterol (FIGS. 5A and 5B).

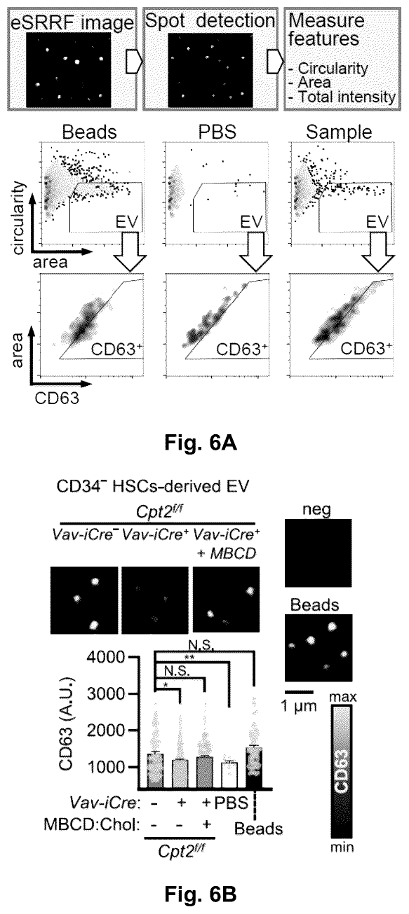

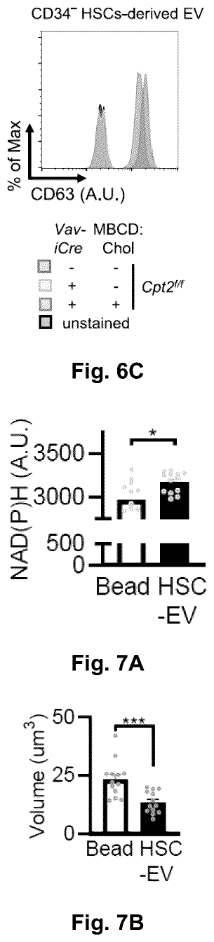

Magnetic enrichment of EVs cannot resolve marker expression for single EVs. Rather, it provides marker expression for the whole EV population. To test the content of EV markers at single EV levels, enhanced super-resolution radial fluctuations (eSRRF) microscopy was employed, which has been demonstrated to resolve structure close to 100 nm. Commercially available fluorescent beads were used, which confirmed that eSRRF imaging is in a resolution domain compatible with EV investigation (data not shown). HSC-derived EVs were immuno-stained for CD63 and imaged using eSRRF microscopy (FIG. 6A). This single-EV technique provides evidence that conditional Cpt2 deletion affects CD63 levels in EVs, which were partially recovered by the replenishment of intracellular cholesterol (FIG. 6B).

Finally, a recently developed high-resolution single-particle platform, NanoFCM NanoAnalyzer (nFCM), was used to investigate the expression of CD63 on HSC-derived EVs. The nFCM analysis confirmed that CD63 loading into EVs is dependent on the FAO-cholesterol axis and that external supplementation of cholesterol can partially recover CD63 loading in FAO-deficient HSCs (FIG. 6C).

### Example 5: HSC-derived EVs, but not EVs Derived from Other Cell Types, Improve HSC Function

### NAD(P)H Analysis by Flow Cytometry

Bone marrow mononuclear cells (BMMNCs) were isolated and stained for surface markers as described above. After staining, samples were subdivided in 3-5 fractions, each of them was incubated for 5 min at 37° C. in 2% FBS-PBS in presence of FCCP 1 mM, or H₂O₂ 100 mM or rotenone 100 nM. After incubation, samples were recorded at LSRII, NAD(P)H autofluorescence was detected in the Indo-Violet channel (excitation laser at 350 nm, band-pass emission 450/50 nm). When indicated, samples exposed with FCCP or H₂O₂ were eventually incubated with H₂O₂ 100 mM or rotenone 100 nM (respectively) at 37° C. for additional 5 min then acquired at LSRII. To compensate for artifact related to cell size, autofluorescence intensity was normalized on FSC-A values, then scaled by multiplying for 104. This index was utilized as a reporter of NAD(P)H concentration. The use of size calibration beads (SPHERO Particle Size Standard Kit #PPS-6K, Spherotech, Lake Forest, IL, USA) confirms that the FSC-A can be used as a readout of cell size.

### Results

Because HSCs actively produce EVs and their biology depends on FAO and cholesterol, the impact of EVs on HSC function was investigated. NAD(P)H levels and mitochondrial mass of CD34⁻ HSCs cultured in the presence of HSC-derived EVs or compensation bead-derived negative controls were assessed.

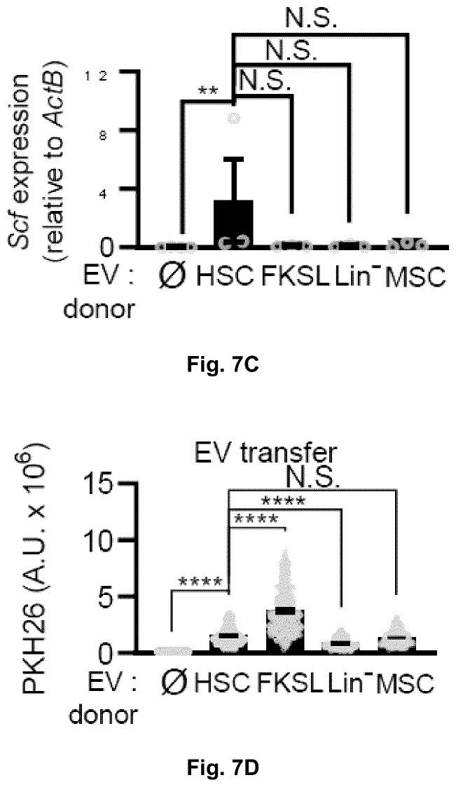

HSC-derived EV exposure elevated NAD(P)H levels in acceptor HSCs while lowering their mitochondrial mass (FIGS. 7A and 7B), two features of mitochondrial physiology that distinguish HSCs from committed progenitors. In addition, the exposure of HSCs to HSC-derived EVs significantly increased the expression of SCF.

Of note, this effect was not observed when EVs were obtained from committed progenitor (FKSL or Lin⁻) cells or mesenchymal stem cells (MSCs) (FIG. 7C), although HSCs do appear to actively take up EVs from these other origins (FIGS. 7C and 7D).

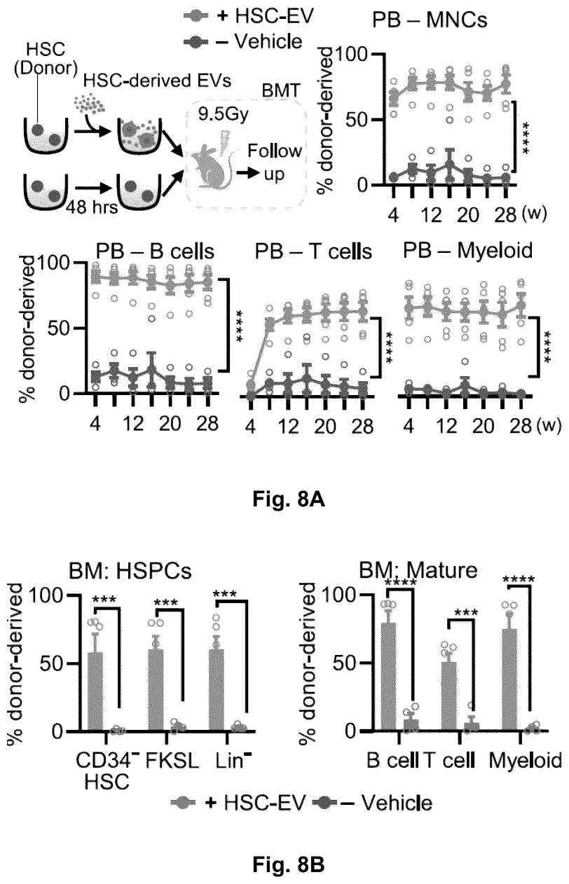

Next, it was examined whether EV supplementation regulated HSC function by measuring the reconstitution potential in vivo. CD34⁻ HSCs were isolated and cultured for 48 h with HSC-derived EVs or vehicle and then subjected to competitive BMT (FIG. 8A, left). Strikingly, HSCs supplemented with EVs displayed a higher capacity to reconstitute the hematopoietic system without lineage bias compared to HSCs receiving vehicle in both peripheral blood (FIG. 8A) and bone marrow (FIG. 8B).

These results confirm that HSCs can stimulate their self-renewal potential in an autocrine manner through EV-mediated signaling.

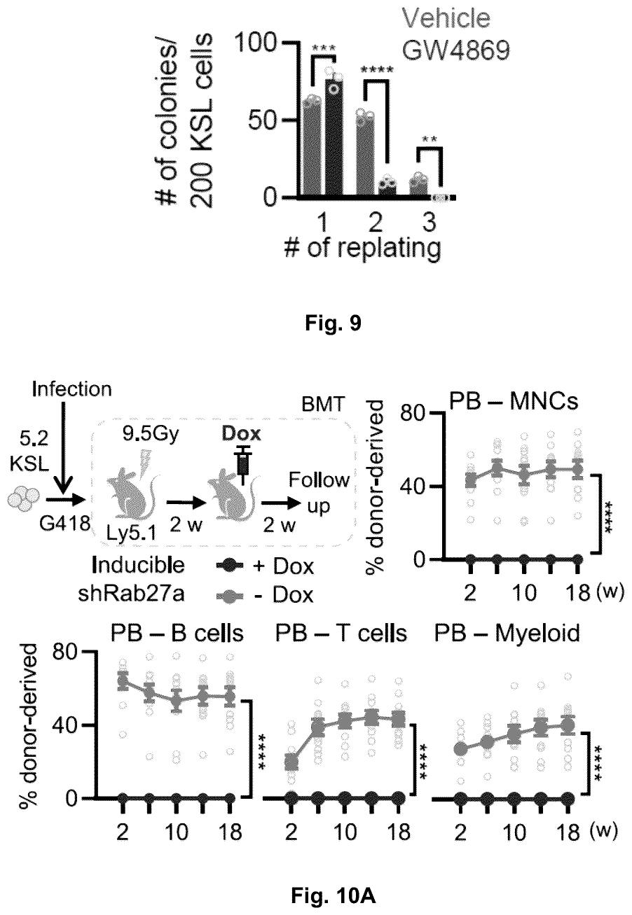

To confirm this new paradigm, EV formation was inhibited in vitro by treating hematopoietic stem and progenitor cells (HSPCs) with GW4869 (inhibitor of neutral sphingomyelinase, widely used to block EV generation) and then measuring their colony-forming capacity upon serial replating. While vehicle-treated HSPCs were able to produce colonies after three re-platings, cells treated with GW4869 only formed colonies in the first two re-platings, indicating loss of self-renewal (FIG. 9).

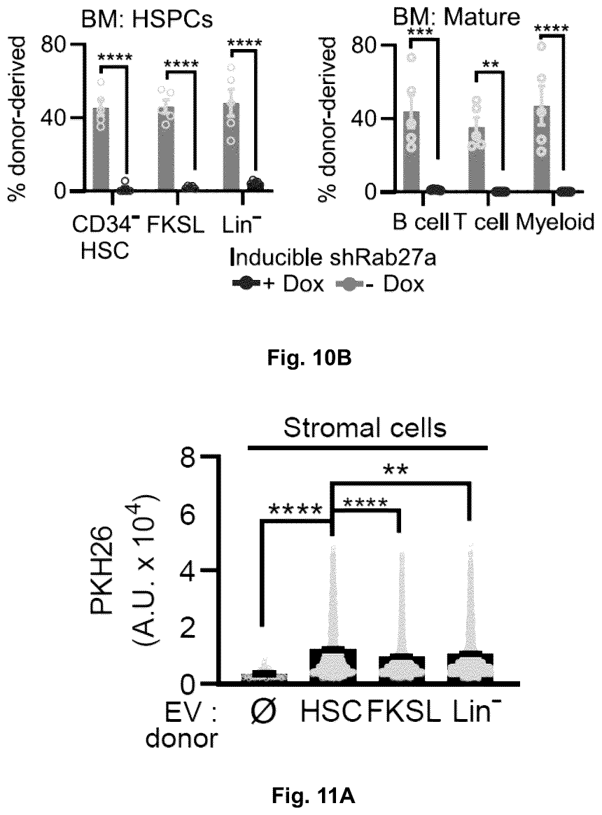

Rab27a regulates EV secretion by promoting multivesicular endosome docking at the plasma membrane. To confirm in vivo the inhibition of EV formation on HSC function, a Dox-inducible short hairpin RNA (shRNA) was used for Rab27a, able to reduce the production of EVs in vitro (data not shown). Sorted FKSL cells were infected in vitro and selected with G418 (1 mg/mL) before undergoing competitive BMT (FIG. 10A, left). Dox induction (performed 2 weeks after BMT) resulted in a dramatic loss of reconstitution potential without lineage bias in both peripheral blood (FIG. 10A) and bone marrow (FIG. 10B) 2 weeks after Dox administration and remained stable up to 18 weeks.

HSCs reside in a special microenvironment in the bone marrow called the HSC niche (or the niche) that maintains hematopoietic homeostasis through interactions between various niche cells/factors and HSCs. The data shown herein show that HSC-derived EVs stimulate HSC function in an autocrine manner. It was further tested whether this process could also involve the participation of supporting stromal cells. To that end, EVs were isolated from HSCs, FKSL cells, and Lin cells stained with PKH26, as described in FIG. 3A. Fluorescently labeled EVs were next administered to bone marrow stromal cells in vitro, and the uptake of EVs was evaluated by fluorescence microscopy.

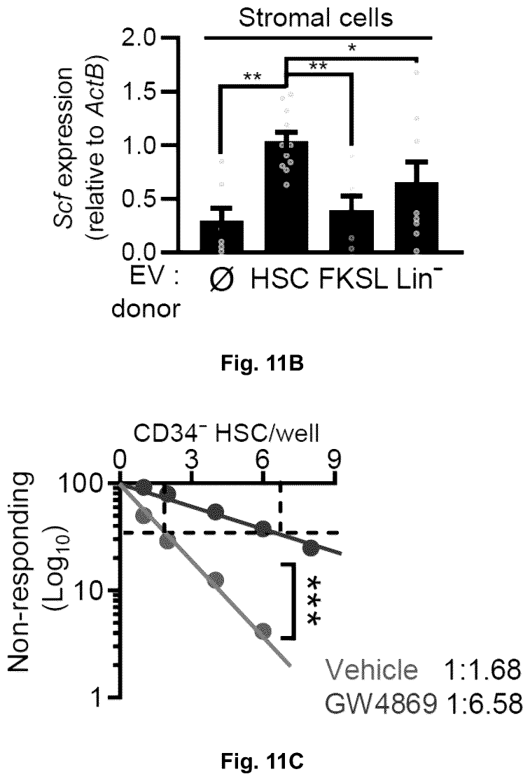

Stromal cells preferentially take up EVs released by HSCs compared to those obtained from committed progenitors (FIG. 11A). Of note, EVs derived from HSCs, but not from committed bone marrow progenitors, were able to potently stimulate SCF expression in stromal cells (FIG. 11B). This indicates that EV-mediated signaling instructs stromal cells to provide a microenvironment favorable for HSC maintenance.

Finally, the colony-forming ability of CD34⁻ HSCs was assessed after 6 weeks of co-culture with stromal cells in the presence of GW4869. It was observed that GW4869 significantly impairs HSC maintenance in vitro, confirming the importance of EV biogenesis in hematopoietic homeostasis (FIG. 11C).

1. A method of expanding a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

2. A method of increasing the expression of stem cell factor (SCF) in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

3. (canceled)

4. A method of increasing NAD(P)H levels in a population of recipient cells comprising recipient HSCs and/or recipient HPCs, the method comprising contacting the population of recipient cells with HSC-derived EVs and/or HPC-derived EVs, wherein the HSC-derived EVs and/or HPC-derived EVs have been substantially isolated from a culture medium, in which a population of donor cells comprising donor HSCs and/or donor HPCs has been cultured.

5. (canceled)

6. (canceled)

7. The method of claim 1, wherein the population of recipient cells is contacted with HSC-derived EVs and/or HPC-derived EVs at a ratio of 1-3×10\hat{ }6 (HSC-derived EVs and/or HPC-derived EVs) per (recipient HSC and/or recipient HPC).

8. The method of claim 1, wherein the step of substantially isolating the HSC-derived EVs and/or HPC-derived EVs from the culture medium is performed by ultracentrifugation, polyethylene glycol (PEG) centrifugation, or size-exclusion chromatography.

9. The method of claim 1, wherein the donor HSCs and/or donor HPCs are obtained from a mammal.

10. The method of claim 9, wherein the donor HSCs and/or donor HPCs are obtained from a human.

11. The method of claim 1, wherein the recipient cells are obtained from a mammal.

12. The method of claim 11, wherein the recipient cells are obtained from a human.

13. The method of claim 1, wherein the recipient cells are recipient HSCs.

14. The method of claim 1, wherein the recipient cells are recipient HPCs.

15. The method of claim 1, wherein the donor cells are donor HSCs.

16. The method of claim 1, wherein the donor cells are donor HPCs.

17. The method of claim 1, wherein the culture medium is based on Iscove's Modified Dulbecco's Medium (MDM), Basal Media Eagle's (BME), Dulbecco's Modified Eagle Medium (DMEM), DMEM/F-12 medium, Roswell Park Memorial Institute (RPMI) medium, RPMI 1640 medium, or Stem Span™ Serum Free Expansion Media (SFEM).

18. The method of claim 1, wherein the culture medium comprises one or more agents selected from the group consisting of thrombopoietin (TPO), stem cell factor (SCF), insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), [[Oct4-activating]] Oct4-activating compound 1 (OAC1), p38 MAPK inhibitor(s), JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS).

19.-30. (canceled)
